(19) United States
(12) Patent Application Publication
(10) Pub. No.: US 2026/0311871 A1
(43) Pub. Date: Oct. 8, 2026

(54) Emotion-Aware AI System with Cyclical Feedback Architecture for Adaptive User Experience.

(71) Applicants: Tinica Walker, Auburn, GA (US); Sean Walker, Auburn, GA (US)

(72) Inventors: Tinica Walker, Auburn, GA (US); Sean Walker, Auburn, GA (US)

(21) Appl. No.: 19/169,170

(22) Filed: Apr. 3, 2025

(51) Int. Cl. G16H 50/20; G06F 3/01; G16H 10/60; G16H 40/67

(52) U.S. Cl. CPC G16H 50/20; G06F 3/012; G06F 3/013; G06F 3/015; G16H 10/60; G16H 40/67; G06F 2203/011

(57) ABSTRACT

The Emotion-Aware AI System is a modular, cross-device framework that detects, classifies, and responds to emotional, somatic, and physiological states in real time. It captures multimodal signals, including facial expressions, vocal tone, heart rate variability, glucose levels, and skin response via sensors, wearables, and APIs. These inputs, combining both external expressions and internal somatic cues, are fused into a unified state vector and classified using deep learning. An embedded SIAOAIR AI Co-Pilot enables agentic intelligence, orchestrating proactive, context-aware actions using reinforcement learning, behavioral feedback, and real-time adaptation with or without user input. Post-intervention data is evaluated locally and securely transmitted (encrypted) to a Federated Aggregation Server for ongoing model refinement. A dedicated privacy layer enforces encryption and differential privacy. Oversight is maintained through an AI Governance and Mission Control layer, ensuring transparency, explainability, and ethical, compliant human-AI interaction.

### BACKGROUND OF THE INVENTION

### Field of the Invention

This invention relates to affective computing, artificial intelligence (AI), and digital health. More specifically, it pertains to systems and methods for real-time recognition of multimodal emotional, somatic, and physiological states, chronic condition monitoring, and context-aware adaptive intervention.

The invention leverages deep learning, reinforcement learning, and privacy-preserving architectures, specifically federated learning to enable personalized classification and agentic intervention across mobile devices, wearables, and computing environments. These systems act proactively and autonomously, adapting in real time to internal bodily cues and environmental context without requiring continuous user input.

### Description of the Related Art

Current emotion-aware systems typically rely on single-modality inputs (e.g., facial recognition or voice analysis) and are often built on rule-based or retrospective logic.

These approaches lack the ability to interpret complex, real-time somatic and physiological variations, such as heart rate variability (HRV), glucose fluctuations, or sleep pattern disruptions and often fail to deliver personalized, adaptive, or agentic responses that proactively support user well-being.

Additionally, most existing systems do not support seamless cross-device coordination, multimodal signal fusion, or compliance with emerging AI governance frameworks such as the EU AI Act, NIST AI Risk Management Framework (AI RMF), or ISO/IEC 42001. These limitations contribute to significant gaps in system accuracy, privacy assurance, explainability, and user trust.

### Problems in the Prior Art

Conventional systems exhibit the following limitations:
Static, non-adaptive logic with no real-time feedback integration or agentic behavior.
Incomplete or inaccurate interpretation of multimodal and somatic signals (e.g., HRV, GSR, glucose variability, posture, and behavioral data).
Absence of embedded explainability tools, fairness auditing mechanisms, or a dedicated AI governance layer.
Lack of federated learning, edge computing, or privacy-by-design architectures to enable data minimization and local intelligence.
No unified framework for integrating emotional, somatic, and physiological inputs into meaningful, context-aware actionable intelligence.

### Solution Provided by the Invention

This invention introduces a modular, multilayered architecture for continuous emotional, somatic, and physiological intelligence across smartphones, wearables, and computing devices.

It captures multimodal signals, including facial expressions, vocal tone, heart rate variability (HRV), blood glucose, respiration, galvanic skin response (GSR), keystroke dynamics, and contextual data, via medical-grade sensors, embedded APIs, and ambient environmental inputs.

A signal processing and fusion engine performs denoising, normalization, and alignment to construct a unified emotional-somatic-health state vector, classified using convolutional neural networks (CNNs), long short-term memory networks (LSTMs), and Transformers. Adaptive interventions are selected in real time through agentic reinforcement learning agents or hybrid rule-based logic.

An embedded SIAOAIR AI Co-Pilot spans Layers 3 through 7, orchestrating state classification, adaptive interventions, cross-device continuity, behavioral feedback monitoring, and personalized recommendations. The system operates with or without explicit user input, demonstrating agentic autonomy. For example, when elevated stress is detected through somatic signals (e.g., HRV drop, breathing irregularity), it may autonomously prompt deep breathing without waiting for user activation.

A continuous feedback engine tracks post-intervention signals such as HRV recovery, emotional stabilization, and glucose rebalancing. Model refinement occurs locally using on-device federated learning with encrypted updates, which are then merged and redistributed to enhance global system intelligence without compromising privacy.

A dedicated AI Governance, Explainability, and Mission Control Layer supervises all system layers, monitoring classification accuracy, model drift, feedback loop stability, and intervention effectiveness. It enables automated alerts, model versioning, and retraining workflows, ensuring ethical, transparent, and resilient performance.

BLE and JSON-based synchronization protocols support seamless emotional-somatic state continuity across devices. Longitudinal dashboards and LLM-generated summaries surface interpretable trends, enabling proactive and personalized emotional health management over time.

### BRIEF SUMMARY OF THE INVENTION

The present invention discloses a modular, multilayered Emotion, Somatic, and Physiology-Aware Artificial Intelligence (AI) system that continuously detects, classifies, and adapts to users'emotional and internal bodily states in real time. Unlike conventional systems limited to static rules or single-modality inputs, this invention integrates multimodal and somatic sensing, including facial expressions, vocal tone, heart rate (HR), heart rate variability (HRV), galvanic skin response (GSR), blood glucose, respiration, keystroke dynamics, posture, and contextual metadata, across mobile devices, wearables, and computing platforms.

### The Architecture Consists of Ten Interconnected Functional Layers

1. Multimodal Sensor Suite
2. Signal Processing & Fusion
3. AI-Based Emotion, Somatic & Health Classification
4. Contextual Adaptive Intervention
5. Cross-Device Integration
6. Feedback Monitoring & Learning
7. Personalized Recommendation & Federated Refinement
8. Longitudinal Reporting & Insight
9. User Preferences & Privacy Governance
10. AI Governance, Explainability & Mission Control

A core SIAOAIR AI Co-Pilot capability spans Layers 3 through 7, orchestrating intelligent classification, agentic intervention, cross-device synchronization, feedback-informed learning, and personalized guidance with or without explicit user input. The system exhibits agentic intelligence, capable of acting autonomously and proactively. For instance, upon detecting elevated stress via HRV decline and GSR spike, it may autonomously suggest a calming activity such as deep breathing without waiting for user activation. Continuous learning occurs via encrypted, on-device federated learning, transmitting only model updates (not raw data) to a secure Federated Aggregation Server for system-wide refinement and redistribution.

A dedicated AI Governance, Explainability & Mission Control Layer provides end-to-end oversight of model integrity, fairness, explainability, and regulatory compliance. It manages anomaly detection, version control, and real-time performance monitoring across all layers. Collectively, these capabilities support proactive well-being, ethical automation, and resilient, adaptive human-AI collaboration.

### BRIEF DESCRIPTION OF THE SEVERAL DRAWINGS

FIG. 1 illustrates a modular AI system that captures and fuses multimodal inputs, visual, auditory, biometric, behavioral, somatic, and contextual into a unified state vector for real-time emotion, physiology, and somatic classification. Adaptive interventions are selected via reinforcement learning and synchronized across devices. The embedded SIAOAIR AI Co-Pilot spans Layers 3-7, orchestrating state interpretation, intervention selection, and continuous adaptation with or without explicit user input.
With input: Users may confirm, reject, or customize suggestions.
Without input: The system exhibits agentic behavior, proactively initiating nudges based on high-confidence patterns.
   Federated learning ensures privacy-preserving model refinement, while a centralized aggregation server merges encrypted updates into a unified adaptive model. An AI Governance, Explainability & Mission Control Layer monitors system integrity, ethical compliance, and operational performance.

FIG. 2 illustrates the real-time detection and feedback refinement loop. Multimodal and somatic inputs are preprocessed, aligned, and classified to trigger adaptive interventions. Post-intervention signals (e.g., HRV recovery, emotional stability) are monitored locally. Federated learning enables secure model updates without transmitting raw data.

FIG. 3 shows a closed-loop system for emotion, somatic, and physiological detection, agentic intervention, and continuous learning. Inputs are classified and routed to the intervention layer, where the SIAOAIR AI Co-Pilot adapts responses autonomously.
The system does not wait for a prompt—it acts proactively.
For example: Upon detecting physiological stress markers, it may suggest deep breathing or a short walk, even if the user is unaware or inactive.
   Federated learning updates are integrated into the unified adaptive model. A parallel reporting layer visualizes trends (e.g., emotional load, recovery patterns) in real time without disrupting core functionality.

FIG. 4 presents a distributed sensing and inference architecture spanning wearables, smartphones, and laptops. It enables cross-device synchronization, collaborative sensing, and decentralized model training. Encrypted model weights from each device node are transmitted to a secure Federated Aggregation Server for merging and global redistribution.

FIG. 5 diagrams the longitudinal reporting and insight engine. It visualizes trends over time, such as HRV recovery, glucose regulation, engagement levels through emotional heatmaps, cognitive strain charts, and LLM-generated summaries. These outputs inform personalized long-term intervention strategies and behavioral coaching.

FIG. 6 depicts a local laptop node operating within the federated learning ecosystem. It performs real-time emotional, physiological, and somatic inference, adaptive UI adjustments, and periodic encrypted model updates. These updates are securely transmitted to the Federated Aggregation Server, supporting continuous system learning and privacy-preserving scalability.

### DETAILED DESCRIPTION OF THE INVENTION

The present invention discloses a modular, multilayered Emotion, Somatic, and Physiology-Aware Artificial Intelligence (AI) system that continuously detects, classifies, and responds to a user's emotional, behavioral, and bodily states in real time. It advances beyond conventional emotion-detection systems by fusing multimodal sensing, adaptive learning, privacy-preserving personalization, and AI governance. Deployed across everyday devices, smartphones, wearables, and laptops, the system operates through ten interlinked layers forming a closed-loop, feedback-driven experience.

### 1. Multimodal Sensor Suite (Input Layer)—See FIGS. 1 & 6

This layer gathers signals across visual, auditory, behavioral, contextual, and somatic domains via medical-grade and consumer-grade devices.
Wearables capture HR, HRV, GSR, respiration, sleep patterns.
Medical sensors add glucose (CGMs), SpO₂, EEG, EMG, and chronic condition markers.
Smartphones/webcams extract facial expressions, voice tone, typing rhythm, gestures.
Contextual signals (e.g., light, time, noise, calendar events) enhance emotional interpretation.
All sensing follows a user-governed privacy protocol with customizable modality permissions.

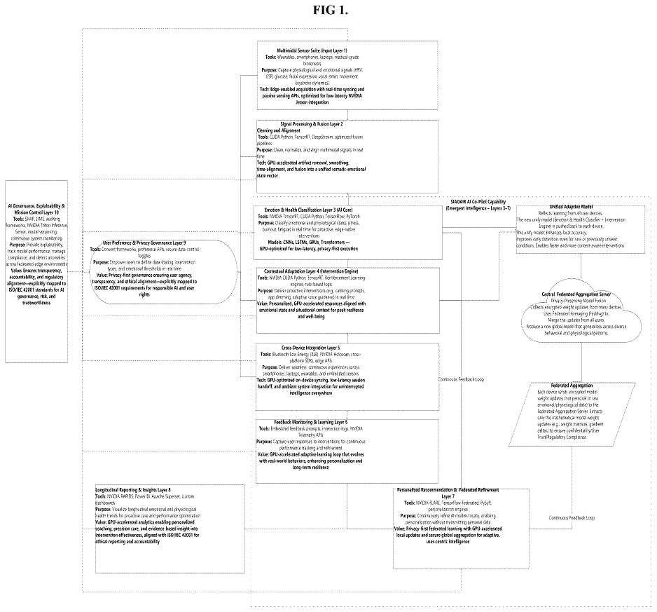

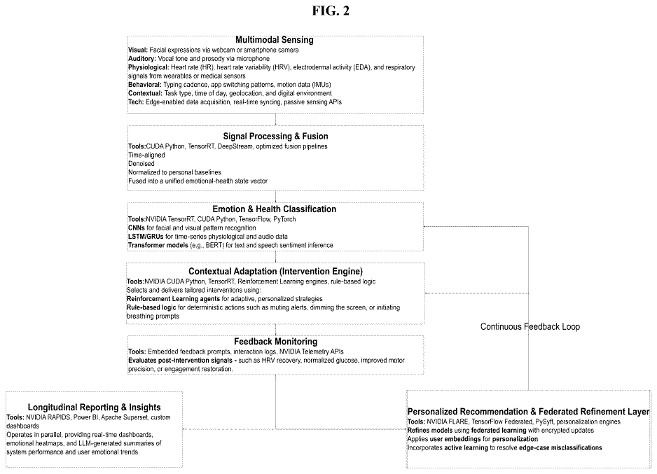

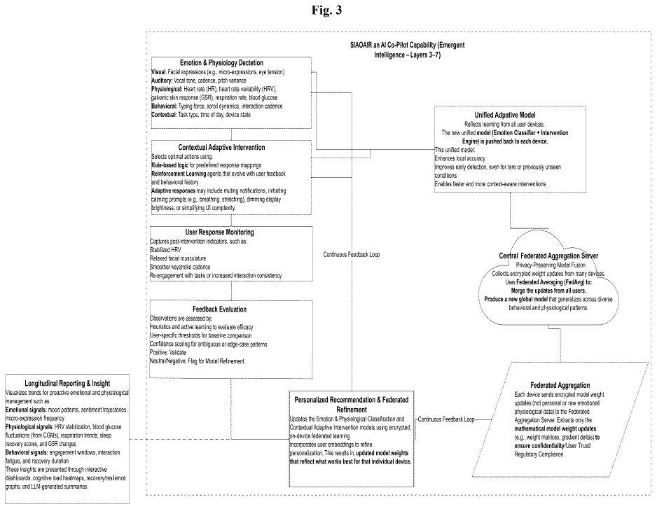

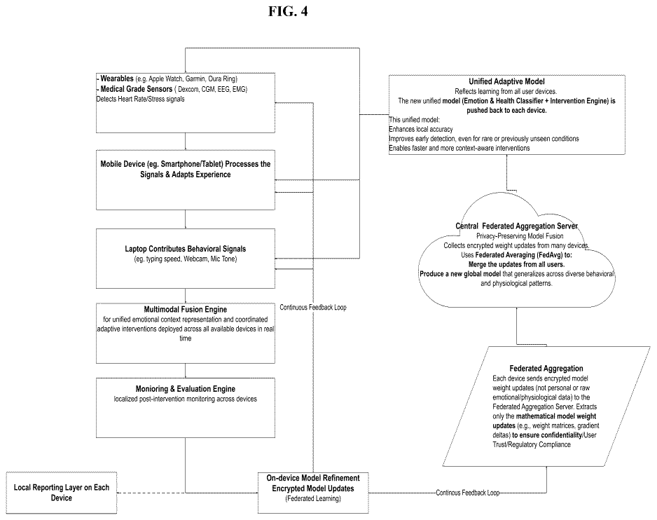

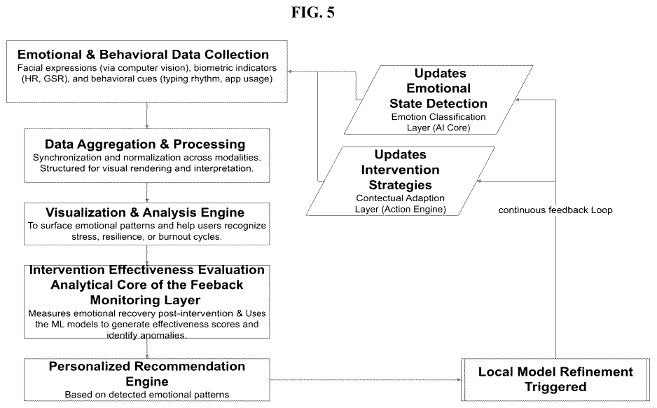

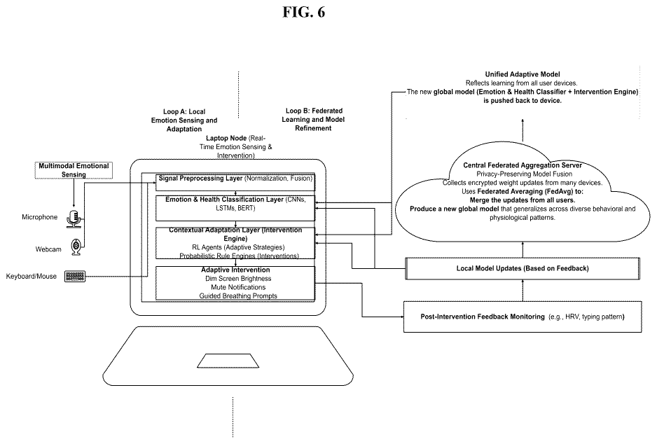

### 2. Signal Processing & Fusion Layer—See FIG. 2

This layer normalizes and aligns data across five modalities: Biometric/Physiological, Behavioral, Visual, Auditory, and Contextual.
Modality-specific noise reduction, baseline normalization, and cross-modal time alignment ensure accuracy.
A fusion engine detects complex patterns e.g., fatigue linked to glucose dip+speech tension+sleep data.
These are encoded into a unified emotional-somatic state vector, powering precise downstream inference.

### 3. Emotion and Health Classification Layer (AI Core)—See FIG. 2

This is the intelligence engine.
The SIAOAIR AI Co-Pilot, embedded across Layers 3-7, orchestrates state classification, feedback monitoring, and personalized recommendations with or without user input, demonstrating agentic, proactive behavior.
A model ensemble handles each modality:
   CNNs for facial features.
   LSTMs/GRUs for time-series biometrics and voice.
   Transformers (BERT, RoBERTa) for emotional context in language.
All models are optimized for on-device inference, preserving privacy while enabling real-time classification.

### 4. Contextual Adaptive Intervention Layer—See FIG. 3

This layer generates real-time, agentic interventions.
Combines reinforcement learning, contextual bandits, and rule-based engines.
Factors in internal signals (e.g., HRV drop, glucose crash) and external context (e.g., time of day, workload).
Example: If burnout is detected, the system dims screens, pauses notifications, or nudges hydration—without a user prompt.
Feedback loops drive continuous personalization over time.

### 5. Cross-Device Integration Layer—See FIG. 4

Enables seamless coordination across devices: smartwatches, phones, laptops, etc.
Uses BLE, Wi-Fi Direct, and mesh networks for low-latency synchronization.
Emotional-somatic state vectors and interventions are shared securely across nodes.
Failover protocols ensure resilience. All interactions are encrypted and governed by user-defined preferences.

### 6. Feedback Monitoring & Learning Layer—See FIGS. 2 & 3

Evaluates intervention outcomes in real time.
Tracks HRV normalization, glucose stabilization, behavioral restoration.
Computes confidence scores for intervention success.
Feeds into federated learning, allowing the system to learn from every user interaction, without compromising privacy.

### 7. Personalized Recommendation & Federated Refinement Layer—See FIG. 2

Generates intelligent next-best actions tailored to the user's emotional-somatic profile.
Uses contextual bandits and user embeddings.
Employs federated learning to refine models on-device and transmit only encrypted weight updates to the cloud, where a unified model is built and shared back.
Supports scalable personalization with full data protection.

### 8. Longitudinal Reporting Layer—See FIG. 5

Provides long-term emotional and physiological insights.
Generates visualizations: stress heatmaps, glucose-emotion overlays, recovery timelines, and resilience scores.
LLMs generate narrative summaries, offering clinician-and user-friendly interpretations.
This analytical layer supports reflective, health-informed decision-making over time.

### 9. User Preferences & Privacy Governance Layer—See FIG. 1

Governs data collection, sharing, and retention.
Users define rules such as:
   “Disable voice sensing after 9 PM”
   “Purge logs every 72 hours”
Implements encryption, access controls, differential privacy, and audit trails.
Fully aligns with HIPAA, GDPR, ISO/IEC 27701, and other global standards.

### 10. AI Governance, Explainability & Mission Control Layer—See FIG. 1

Provides centralized oversight of fairness, explainability, and regulatory compliance.
Monitors classification accuracy, feedback integrity, model drift, and real-time system behavior.
Maintains transparent, accountable, and ethical AI operation, ensuring trust and resilience in every interaction.

### Key Capabilities of the AI Governance, Explainability & Mission Control Layer

### Explainable AI (XAI)

Integrates SHAP and LIME frameworks to generate transparent, human-interpretable justifications for each AI output. This enables users, developers, and auditors to trace the decision-making logic behind emotional, somatic, and physiological classifications and interventions.

### Bias Detection & Fairness Auditing

Continuously monitors algorithmic outputs across demographic and behavioral cohorts to ensure outcome parity. If disparities or drift are detected, the system automatically initiates alerts, triggers retraining workflows, or adjusts model weights to mitigate bias and uphold fairness.

### Federated Audit Logging

Maintains privacy-preserving, tamper-evident logs capturing key lifecycle events, including inference triggers, classification decisions, model updates, and agentic interventions, without exposing raw user data. This supports compliance and traceability while protecting user confidentiality.

### Mission Control & AI Operations (AI Ops)

Provides centralized, real-time oversight for all deployed models and system components. Monitors:
classification accuracy
Model drift
Feedback loop stability
Intervention performance
   Mission Control enforces version control, anomaly detection, rollback safeguards, and retraining protocols to maintain system-wide performance integrity.

### Compliance by Design

The layer is architected to meet global AI governance standards including:
EU Artificial Intelligence Act (EU AI Act)
NIST AI Risk Management Framework (AI RMF)
ISO/IEC 42001 for AI Management Systems
   These standards are embedded into the design to support trustworthy, lawful, and ethically-aligned operations from the ground up.

### Centralized Oversight and Ethical Command Layer

This layer functions as the ethical command center and mission control of the invention. Beyond explainability and regulatory alignment, it delivers holistic oversight of real-time system behavior across all functional layers. It continuously evaluates:
The accuracy of emotional, physiological, and somatic state classifications
The effectiveness and timing of agentic interventions
The integrity of the feedback loop
Signals of drift or degradation

Automated alerts, model versioning, and retraining pipelines are triggered when anomalies or inconsistencies are detected. By providing complete operational transparency, this layer ensures:
Ethical alignment
Long-term reliability
Scalable oversight
System trustworthiness across distributed, real-world environments

### Technology Stack

### 1. Programming Languages

Python—Core language for AI/ML pipelines, federated learning orchestration, signal processing, and system-level automation.
Swift—Native integration for iOS devices, enabling real-time sensor data capture, adaptive interventions, and seamless user interaction on Apple platforms.
Kotlin—Native Android support for edge inference, UI responsiveness, and on-device behavior modeling.

### 2. AI/Machine Learning Frameworks

TensorFlow Lite, PyTorch Mobile, ONNX—Optimized for lightweight, cross-platform deployment on mobile, wearable, and embedded devices. These frameworks support multimodal inference and on-device learning from emotional, somatic, and physiological inputs.

### 3. Edge & Embedded Computing

Mobile Neural Engines—Apple Neural Engine (ANE) and Android Neural Networks API (NNAPI) enable ultra-low-latency inference for real-time classification and agentic intervention.
Edge TPUs & ARM Cortex Processors—Hardware acceleration for processing high-frequency somatic signals from wearables and biosensors.
IoT Edge Platforms—Integration with embedded medical-grade sensors (e.g., CGMs,

HRV monitors), ambient sensing modules, and distributed edge nodes across the user environment.

### 4. Privacy, Governance & Explainability

Federated Learning—Enables on-device training and encrypted model weight sharing across distributed nodes without transmitting raw data. Supports personalization at scale.
Differential Privacy—Applies noise injection to protect individual-level signal contributions while preserving model utility in aggregate analysis.
Consent Management APIs—User-governed access control for sensor activation, data retention, and cross-device sharing. Enables real-time data governance aligned with user intent.
AI Governance Engines—Ensure compliance with global standards (e.g., EU AI Act, NIST AI RMF, ISO/IEC 42001) via embedded audit trails, model versioning, and bias detection mechanisms.
Explainability Tools—SHAP, LIME, and Captum provide per-instance model interpretation, supporting transparency in how emotional-somatic decisions are made and justified.

### Emotion Use Case Scenarios

### 1. Fitness & Health Optimization

Scenario: During physical activity, SIAOAIR detects elevated emotional and somatic stress signals (e.g., HR spikes, facial tension) via biometric and visual inputs. It autonomously triggers adaptive prompts such as breath pacing, intensity modulation, or motivational audio. Recovery trends are tracked post-session to tailor future routines.

Why It Matters: Optimizes performance without overexertion; improves emotional regulation during exertion.

Who Benefits: Athletes, wellness users, physical therapy patients.

### 2. Cognitive Focus & Learning Support

Scenario: During deep work or learning, SIAOAIR detects signs of cognitive fatigue, scrolling loops, facial micro-tension, reduced typing accuracy. It suggests micro-breaks, ambient adjustments, or pacing changes.

Why It Matters: Prevents overload and improves attention sustainability.

Who Benefits: Students, neurodivergent users, professionals in cognitive-demand roles.

### 3. Communication & Emotional Coaching

Scenario: In live conversations or written exchanges, voice pitch escalation or typing aggression triggers real-time nudges or post-interaction emotional feedback.

Why It Matters: Encourages emotional awareness and tone calibration.

Who Benefits: Remote teams, leaders, therapy clients.

### 4. Workplace Wellness & Burnout Prevention

Scenario: Task switching slows, facial fatigue increases, or erratic digital behavior emerges. SIAOAIR initiates proactive wellness actions: break suggestions, async check-ins, or pacing shifts.

Why It Matters: Reduces chronic stress and improves long-term output.

Who Benefits: Remote workers, team leads, HR professionals.

### 5. Positive Emotion Amplification

Scenario: SIAOAIR detects positive affect (e.g., smile, calm voice, balanced HRV) and reinforces with micro-celebrations: animations, sound cues, affirmations.

Why It Matters: Reinforces well-being and builds resilience.

Who Benefits: All users, particularly in recovery or emotional growth.

### 6. Conflict Recovery & Emotional Cooldown

Scenario: After emotional spikes (e.g., increased GSR, HRV suppression, vocal tension), SIAOAIR initiates a cooldown protocol—dimming screens, silencing notifications, suggesting grounding techniques.

Why It Matters: Supports fast emotional recovery post-conflict.

Who Benefits: High-pressure workers, caregivers, frontline staff.

### 7. Social Engagement & Empathy Cues

Scenario: During group or virtual interactions, SIAOAIR detects emotional disengagement (e.g., muted tone, gaze aversion) and nudges re-engagement through empathy prompts or feedback.

Why It Matters: Improves emotional intelligence in digital contexts.

Who Benefits: Team leaders, customer service, neurodivergent users.

### 8. Emotionally Tuned Content Delivery

Scenario: Based on real-time emotion sensing (e.g., boredom, frustration), SIAOAIR adapts the format, tone, or length of digital content to match user emotional capacity.

Why It Matters: Enhances content relevance while minimizing fatigue.

Who Benefits: Learners, streaming consumers, content platforms.

### Condition-aware Use Case Scenarios

### 1. Diabetes Management

Scenario: Detects glucose fluctuations from CGMs and correlates with stress levels or mood indicators. Suggests hydration, food intake, or mindfulness to stabilize glycemic patterns.

Why It Matters: Emotional stress is a critical trigger for glucose dysregulation.

Who Benefits: Type 1 & 2 diabetics, caregivers, endocrinologists.

### 2. Cardiovascular Health

Scenario: SIAOAIR monitors HRV, blood pressure, and resting HR for emotional-cardiac triggers. Suggests calm-inducing exercises at risk thresholds.

Why It Matters: HRV is a primary predictor of cardiac strain.

Who Benefits: Cardiac patients, clinicians, wellness providers.

### 3. Respiratory Conditions (Asthma, COPD)

Scenario: Tracks respiratory rate, SpO₂, and emotional distress. When early signs emerge, it suggests position adjustments, rest, or alerts caregivers.

Why It Matters: Prevents emotional triggers from exacerbating respiratory episodes.

Who Benefits: Asthma/COPD patients, respiratory therapists.

### 4. Epilepsy Risk Detection

Scenario: Detects electrodermal activity spikes, HR surges, or EMG muscle tension that may precede seizures. Initiates automated safety protocols.

Why It Matters: Enables preemptive action through early detection.

Who Benefits: People with epilepsy, families, neurologists.

### 5. Neurodivergence (ADHD/Autism)

Scenario: Monitors gaze patterns, typing rhythm, and switching behavior to identify overload or dysregulation. Adjusts interfaces or offers focus tools.

Why It Matters: Enhances emotional comfort in digital environments.

Who Benefits: Neurodivergent individuals, accessibility teams, educators.

### 6. Parkinson's & Neurodegenerative Monitoring

Scenario: Detects motor changes, voice tremors, or facial micro expression shifts. Flags emotional distress or disease progression trends.

Why It Matters: Links emotional and physical symptoms in early-stage tracking.

Who Benefits: Patients, neurologists, caregivers.

### 7. Anxiety & Panic Support

Scenario: During acute stress, SIAOAIR detects shallow breathing, HRV drops, and vocal tension. Deploys grounding cues or ambient de-escalation.

Why It Matters: Provides in-the-moment relief and builds self-regulation habits.

Who Benefits: Anxiety sufferers, therapists, wellness platforms.

### 8. Depression Monitoring

Scenario: Identifies behavioral withdrawal, slowed activity, reduced expression, late-night screen use and offers gentle nudges or check-ins.

Why It Matters: Facilitates early detection and daily engagement.

Who Benefits: Users with depression, digital mental health tools.

### 9. Burnout & Chronic Stress

Scenario: Monitors disengagement signals, HRV suppression, and task avoidance. Suggests pause, boundary setting, or restorative actions.

Why It Matters: Supports long-term emotional sustainability.

Who Benefits: Knowledge workers, wellness leaders, digital therapists.

### 10. Sleep Health

Scenario: Tracks sleep cycles, HRV recovery, and pre-sleep emotional states. Recommends adjustments to improve nighttime recovery.

Why It Matters: Sleep is tightly coupled with emotional resilience.

Who Benefits: Individuals with insomnia, apnea, or disrupted sleep.

### 11. Hormonal & Postpartum Support

Scenario: Detects mood instability, vocal tension, and HRV fluctuation. Suggests tracking, support prompts, or calming interventions.

Why It Matters: Emotional shifts signal hormonal imbalance or postpartum changes.

Who Benefits: Women, mothers, clinicians, wellness apps.

### 12. Autoimmune & Chronic Illness

Scenario: Tracks fatigue, movement reduction, and thermoregulation to correlate emotional strain with autoimmune flare risks.

Why It Matters: Helps identify emotional triggers in chronic inflammation cycles.

Who Benefits: Patients with Lupus, MS, and other autoimmune disorders.

### Potential Industry Applications

The disclosed Emotion-Aware AI system is designed for cross-industry deployment where real-time emotional, somatic, and physiological intelligence can drive meaningful improvements in health, performance, user experience, and safety. By dynamically interpreting signals such as facial expressions, vocal tone, heart rate variability (HRV), blood glucose, respiratory rate, electrodermal activity (EDA), and movement patterns, the system delivers agentic, context-aware responses without requiring user input.

### Healthcare

Used in digital therapeutics, mental health platforms, and chronic disease management.
Fuses emotional context with physiological markers (e.g., CGM glucose trends, HRV, EDA)
Detects stress-induced glycemic variability, cardiovascular strain, and emotional volatility
Enables early intervention and adaptive, personalized care
   Impact: Prevents escalation, supports emotional regulation, improves outcomes

### Workplace Productivity

Integrated into wellness platforms and digital workspaces.
Monitors HRV suppression, reduced typing cadence, and emotional stress
Adjusts workload pacing, interface elements, or suggests breaks
   Impact: Reduces burnout, sustains performance, and promotes employee well-being

### Education

Applied in responsive learning environments.
Detects cognitive overload and disengagement through biometric and behavioral signals (e.g., respiration, pupil dilation)
Dynamically adapts content delivery and supports neurodivergent learners
   Impact: Improves learning retention, focus, and accessibility

### Consumer Technology

Embedded in smartphones, wearables, laptops, and smart environments.
Adapts UI complexity, notification timing, and ambient lighting in response to real-time signals like facial strain or vocal fatigue
   Impact: Delivers emotionally intelligent, user-aware digital experiences

### Gaming & Entertainment

Enables adaptive storytelling and gameplay experiences.
Responds to emotional states (e.g., frustration, joy) and physiological signals (e.g., HRV, body tension)
Modulates difficulty, pacing, or narrative accordingly
   Impact: Increases immersion, personalization, and therapeutic gaming potential

### Transportation

Deployed in vehicles to monitor driver well-being.
Tracks HRV, respiration, and motion to detect fatigue or stress
Triggers adaptive features like seat adjustments, ambient lighting, or alerts
   Impact: Enhances driver safety, comfort, and responsiveness

### Retail & Customer Experience

Used in adaptive digital storefronts and in-store experiences.
Analyzes real-time emotional and biometric signals to personalize engagement
Modifies product recommendations and interface flow based on stress or interest
   Impact: Improves satisfaction, conversion, and loyalty

### Social Media & Communication

Supports emotionally aware messaging and digital wellness.
Monitors emotional arousal (via voice tone, typing rhythm) and somatic stress (e.g., EDA)
Prompts reflective interactions or pauses during moments of distress
   Impact: Promotes healthier digital interactions and reduces online harm

### Financial Services

Integrated into fintech platforms to manage stress-related decision-making.
Detects emotional reactivity and physiological tension during high-stakes actions
Simplifies flows, delays irreversible steps, or provides calming nudges
   Impact: Enhances decision quality, user trust, and behavioral safety

### Scalable Architecture

The system's modular, privacy-first, and cross-device design enables adaptation to sector-specific regulatory, technical, and UX demands.
Federated learning and differential privacy ensure compliance
Agentic AI delivers proactive support tailored to individual needs
   Impact: Enables industry-wide deployment of emotional and physiological intelligence at scale

### Key Advantages

### Real-Time, Context-Aware Emotional and Physiological Intelligence

Continuously detects and interprets stress, fatigue, and metabolic load within milliseconds, across devices by fusing multimodal signals such as HRV, electrodermal activity (EDA), facial micro-expressions, and vocal tone. Enables timely, situationally aware responses.

### Adaptive Personalization Via Biometric Feedback Loops

Learns from user-specific recovery signals and longitudinal behavior patterns to optimize predictions and interventions. Federated learning ensures hyper-personalized adaptation while preserving on-device privacy.

### Enhanced Wellbeing and Cognitive Resilience

Supports emotional regulation and physiological recovery through real-time, context-triggered nudges, ranging from breathwork cues and screen dimming to hydration prompts and environmental adjustments.

### Granular User Control and Transparent Data Governance

Empowers users with precise control over sensing, intervention preferences, and data flow.

Features include opt-in modules, real-time privacy dashboards, customizable sensing rules, and auto-deletion settings.

### Seamless Cross-Device Synchronization of Emotional and Physiological Context

Maintains a persistent, synchronized understanding of the user's state across smartphones, wearables, tablets, and laptops using secure mesh networking and session-aware intelligence.

### Comprehensive Multimodal Analytics

Transforms complex signals into intuitive insights—such as emotion-glucose overlays, stress heatmaps, and recovery timelines—delivered through LLM-generated summaries for individuals, caregivers, and enterprise stakeholders.

### Emotionally Sensitive and Ethically Grounded Design

All interventions are guided by user-defined risk thresholds, emotional context, and consent protocols. Prioritizes dignity, human-in-the-loop safety, and context-aware responsiveness—especially in sensitive scenarios.

### Mission Control Under Ai Governance

A centralized AI Ops module oversees real-time performance, anomaly detection, model versioning, and safety monitoring—ensuring stable, ethical, and transparent operations at scale.

### SIAOAIR AI Co-Pilot Capabilities

Functions as an intelligent, agentic co-pilot—capable of sensing, interpreting, and acting without explicit user commands. Delivers proactive, personalized nudges, adjusts cognitive loads, and orchestrates wellness actions using emotional, contextual, and biometric inputs.

### Governance-by-Design With Systems Thinking & Poka-Yoke Architecture

Complies with global AI standards (EU AI Act, NIST AI RMF, ISO/IEC 42001) and integrates explainability (SHAP, LIME), fairness auditing, and federated audit trails. Built using systems thinking and poka-yoke safeguards to prevent failure, support ethical escalation, and ensure long-term resilience.

### CONCLUSION

The disclosed Emotion-Aware AI system, SIAOAIR, embeds the heartbeat of AI into a modular, cross-device architecture that unifies emotional and physiological sensing, adaptive interventions, federated learning, and real-time governance. It advances affective computing from passive recognition to agentic, proactive, and context-aware care across smartphones, wearables, tablets, and desktops.

SIAOAIR functions as a somatic, intelligent co-pilot, sensing the subtle rhythms of the body and mind. It interprets user states and autonomously delivers timely, precision-tuned interventions with or without user input. Real-time adaptation is driven by reinforcement learning and personalized baselines that evolve as the system learns. Federated refinement ensures model updates are encrypted and privacy-preserving, enabling continuous evolution without exposing raw data.

At its core lies the AI Mission Control Layer, SIAOAIR's ethical compass. This centralized governance hub integrates explainability, anomaly detection, model versioning, and compliance with evolving global standards (EU AI Act, NIST AI RMF, ISO/IEC 42001). Designed with systems thinking and poka-yoke principles, the platform safeguards integrity, resilience, and accountability by design.

SIAOAIR doesn't just process signals, it feels the pulse of user experience. It is the living rhythm of AI: sensing, interpreting, acting, observing, adapting, improving, and reporting. This human-centered foundation redefines intelligent automation, making emotional wellness, cognitive clarity, and ethical AI not just possible, but inevitable.

### TECHNICAL GLOSSARY

### Active Learning

An AI approach that prioritizes uncertain or misclassified data points for retraining.

System Role: Improves model accuracy by focusing on low-confidence classifications.

Applied In: Feedback Monitoring, Model Refinement Layer

Figure(s): FIGS. 2, 3, 5

### Agentic Intelligence

A system's capacity to autonomously sense, interpret, and respond to user needs or environmental cues—acting without explicit prompts.

System Role: Enables proactive, self-directed decision-making and action.

Applied In: SIAOAIR Co-Pilot, Contextual Adaptive Intervention Layer

Figure(s): FIGS. 1, 2, 3, 6

### API—Application Programming Interface

A set of routines and protocols enabling communication between software systems.

System Role: Connects sensor hardware and software modules.

Applied In: Multimodal Sensor Suite, Adaptive Intervention Layer

Figure(s): FIGS. 1-6

### BERT—Bidirectional Encoder Representations From Transformers

A transformer-based deep learning model used for NLP and emotion classification from text.

System Role: Sentiment and emotional tone detection.

Applied In: Emotion Classification Layer

Figure(s): FIGS. 1, 2, 6

### BLE—Bluetooth Low Energy

A short-range wireless protocol used for power-efficient communication between devices.

System Role: Enables secure, real-time emotional context synchronization across devices.

Applied In: Cross-Device Integration Layer

Figure(s): FIGS. 1, 4

### CGM—Continuous Glucose Monitor

A medical-grade device that tracks blood glucose (sugar) levels in real time.
Function: Measures interstitial glucose levels continuously (every few minutes).
Common Devices: Dexcom G7, Abbott FreeStyle Libre, Medtronic Guardian.
Data Access: Sends glucose data to smartphones, smartwatches, or cloud platforms via Bluetooth.

### CNN—Convolutional Neural Network

A type of AI model that helps computers understand images by finding patterns like shapes and edges.

System Role: Classifies facial expressions.

Applied In: Emotion Classification Layer

Figure(s): FIGS. 1, 2, 6

### Contextual Bandits—Contextual Bandit Algorithms

A machine learning method for real-time decision-making with partial feedback.

System Role: Recommends next-best adaptive actions under uncertainty.

Applied In: Personalized Recommendation Layer

Figure(s): FIGS. 2, 3

### EDA—Electrodermal Activity

A physiological signal that measures the skin's electrical conductance, which varies with sweat gland activity. It is commonly used as an indicator of emotional arousal or stress.

### Edge AI

Running AI algorithms directly on devices (e.g., laptops, wearables) without relying on cloud computing.

System Role: Improves privacy and real-time responsiveness.

Applied In: All Edge Layers

Figure(s): FIGS. 1, 6

### EMG—Electromyography

A method used to measure the electrical activity produced by skeletal muscles. it detects muscle activation signals.

### Federated Learning

A privacy-preserving machine learning approach where models are trained locally and aggregated without sharing raw data.

System Role: Refines Emotion Classification and Intervention Logic across distributed devices.

Applied In: Federated Model Refinement Layer

Figure(s): FIGS. 1, 2, 3, 4, 6

### GRU—Gated Recurrent Unit

A variant of RNNs used for time-series prediction.

System Role: Interprets biometric and vocal signals for emotional analysis.

Applied In: Emotion Classification Layer

Figure(s): FIGS. 1, 2, 6

### GSR—Galvanic Skin Response

A measure of skin conductance linked to arousal or stress.

System Role: Biometric indicator of emotional state.

Applied In: Multimodal Sensor Suite, Feedback Monitoring Layer

Figure(s): FIGS. 1, 2, 4, 5

### Heuristics—Heuristic Evaluation

Rule-based methods for fast evaluation and decision-making.

System Role: Assess effectiveness of interventions.

Applied In: Feedback Monitoring & Contextual Adaptation

Figure(s): FIGS. 2, 3, 5

### HR—Heart Rate

The number of heart beats per minute, indicating arousal or rest.

System Role: Biometric emotional input.

Applied In: Sensor Suite, Feedback Monitoring

Figure(s): FIGS. 1, 2, 3, 4, 5, 6

### HRV—Heart Rate Variability

The variance in intervals between heartbeats; low HRV is linked to stress.

System Role: Indicator of stress, fatigue, and emotional recovery.

Applied In: Sensor Suite, Feedback Monitoring Layer

Figure(s): FIGS. 1, 2, 3, 4, 5, 6

### IMU sensors—Inertial Measurement Units

Are a critical component in motion sensing and are especially useful for tracking physical behavior, posture, and fine motor changes.

### JSON—JavaScript Object Notation

Lightweight data-interchange format used for structuring emotional signals and transmitting them between layers or devices.

System Role: Used for cross-device and layer-based communication.

Applied In: Cross-Device Integration, Reporting Layer

Figure(s): FIGS. 1, 5

### LIME—Local Interpretable Model-Agnostic Explanations

A method for explaining the predictions of black-box models.

System Role: Enables model transparency and human interpretability.

Applied In: AI Governance & Explainability Layer

Figure(s): FIG. 1

### LLM—Large Language Model

Advanced NLP model trained on large corpora.

System Role: Generates summaries of emotional insights and trends.

Applied In: Longitudinal Reporting Layer

Figure(s): FIGS. 1, 2, 5

### LSTM—Long Short-Term Memory

A type of RNN designed to retain temporal information.

System Role: Classifies sequential biometric/emotional data.

Applied In: Emotion Classification Layer

Figure(s): FIGS. 1, 2, 6

### ML Ops—Machine Learning Operations

Tools and practices for deploying, monitoring, and updating ML models.

System Role: Supports secure deployment and update of federated models.

Applied In: Federated Refinement Infrastructure

Figure(s): FIG. 6

### Multimodal Fusion—Multimodal Data Fusion

The integration of input signals from multiple modalities (e.g., visual, auditory, biometric).

System Role: Produces a unified emotional state representation.

Applied In: Signal Processing and Fusion Layer

Figure(s): FIGS. 1, 2, 6

### NIST AI RMF—National Institute of Standards and Technology AI Risk Management Framework

A U.S. regulatory framework for responsible AI use.

System Role: Guides risk management, transparency, and fairness.

Applied In: AI Governance & Explainability Layer

Figure(s): FIG. 1

### NLP—Natural Language Processing

A subfield of AI focused on understanding human language.

System Role: Used in emotion detection from text and speech.

Applied In: Emotion Classification Layer

Figure(s): FIGS. 1, 2, 6

### Prosody—Speech Prosody

Rhythmic and tonal characteristics of speech that signal emotion.

System Role: Interprets vocal emotional tone.

Applied In: Sensor Suite, Emotion Classification Layer

Figure(s): FIGS. 1, 2, 4, 6

### RL—Reinforcement Learning

A machine learning approach where agents learn optimal actions via feedback.

System Role: Personalizes interventions based on success history.

Applied In: Contextual Adaptive Intervention Layer

Figure(s): FIGS. 1, 2, 3, 6

### RoBERTa—Robustly Optimized BERT Approach

A variant of BERT trained with more data and longer sequences. Commonly used for emotion classification in text-based analysis.

System Role: Text-based sentiment and emotional classification.

Applied In: Emotion Classification Layer

Figure(s): FIGS. 1, 2, 6

### SDK—Software Development Kit

A toolset for developing software applications.

System Role: Integrates APIs and sensor functionalities.

Applied In: Multimodal Sensor Suite

Figure(s): FIGS. 1, 4

### Sensor Hub Manager (SHM)

A hardware or software layer that aggregates and preprocesses sensor signals before feeding them into the AI engine.

System Role: Pre-processes signals from wearables like Apple Watch.

Applied In: Multimodal Sensor Suite

Figure(s): FIGS. 1, 4

### SHAP—SHapley Additive exPlanations

A technique for explaining individual predictions of AI models.

System Role: Supports explainability and fairness auditing.

Applied In: AI Governance Layer

Figure(s): FIG. 1

### Somatic Intelligence

The interpretation of physical bodily signals such as posture, gesture, muscular tension, and movement as proxies for emotional or physiological states.

System Role: Enhances emotional inference through embodied behavioral cues.

Applied In: Multimodal Sensor Suite, Emotion & Physiology Classification Layer

Figure(s): FIGS. 1, 2, 4, 6

### SpO₂—Peripheral Capillary Oxygen Saturation

It's the estimated percentage of oxygen-saturated hemoglobin in your blood relative to the total hemoglobin that can carry oxygen. It tells you how well oxygen is being transported to parts of your body, especially vital organs like the brain and heart.

### Temporal Alignment—Temporal Data Synchronization

The process of aligning time-stamped data across modalities.

System Role: Ensures coherence in emotional inference.

Applied In: Signal Processing and Fusion Layer

Figure(s): FIGS. 1, 2, 6

### Transformer Models

AI models based on attention mechanisms for sequence data (e.g., BERT, RoBERTa).

System Role: Classify emotion from language-based inputs.

Applied In: Emotion Classification Layer

Figure(s): FIGS. 1, 2, 6

### User Embedding—User Embedding Models

Vector representations of users derived from behavioral/emotional history.

System Role: Powers hyper-personalized emotional predictions and interventions.

Applied In: Personalized Recommendation & Model Refinement

Figure(s): FIGS. 2, 3, 5

### UX—User Experience

The quality of interaction between users and the system.

System Role: Improved through responsive, emotionally intelligent feedback loops.

Applied In: Across all layers

Figure(s): FIGS. 1-6

1. A system for detecting, evaluating, and adaptively responding to a user's emotional and physiological states during ongoing interaction, comprising:
a) two or more user devices operating in coordination, selected from the group consisting of a smartwatch, smartphone, tablet, and laptop, wherein at least one of the two or more user devices is a smartwatch configured to capture biometric data, and at least one other of the two or more user devices is a smartphone, tablet, or laptop configured to capture behavioral, visual, or auditory data;
b) a multimodal sensor suite configured to capture signals across five modalities:
I. Biometric/Physiological: including heart rate variability (HRV), galvanic skin response (GSR), respiratory rate, blood oxygen saturation (SpO2), skin temperature, blood glucose level, and sleep cycle data;
II. Behavioral: including keystroke dynamics, typing rhythm, application usage patterns, scrolling or clicking behavior, and physical movement patterns;
III. Visual: including facial expressions, micro-expressions, gaze tracking, blink rate, and facial muscular tension via integrated or peripheral cameras;
IV. Auditory/Voice: including vocal tone, pitch, cadence, pauses, tremors, and breathing irregularities via one or more microphones;
V. Contextual/Environmental: including time of day, geolocation, ambient light and sound levels, device usage frequency, calendar events, and interaction context;
c) a signal processing and fusion engine configured to normalize, denoise, temporally align, reconcile redundant signals, and synthesize the captured inputs into a unified physiological state vector;
d) an emotion and health classification engine comprising one or more machine learning models selected from convolutional neural networks (CNNs), long short-term memory networks (LSTMs), gated recurrent units (GRUs), and transformer-based architectures, the engine configured to infer user states from the synthesized vector;
e) a contextual adaptive intervention engine configured to deploy one or more adaptive actions based on the inferred state using reinforcement learning or rule-based logic;
f) a monitoring module configured to collect post-intervention signals, including physiological recovery indicators and behavioral patterns;
g) a feedback evaluation engine configured to assess intervention effectiveness using recovery signals, heuristic patterns, and user-specific thresholds, and to adapt intervention strategies based on real-time physiological and behavioral feedback;
h) a personalized recommendation and orchestration module configured to use contextual bandits, reinforcement learning models, and ranking frameworks to interpret the user's physiological and behavioral state, contextual inputs, and user embeddings, and to autonomously trigger next-best actions across the smartwatch, smartphone, tablet, and laptop with or without explicit user commands;
i) a federated refinement engine configured to perform on-device model training and transmit encrypted model weight updates to a federated aggregation server, where updates from multiple devices are merged and redistributed as a global model, without transmitting raw sensor data;
j) a longitudinal reporting and insight module configured to generate dashboards and natural language summaries, including trend visualizations of emotional and physiological states, intervention outcomes, and resilience metrics, optionally via large language models (LLMs);
wherein the two or more user devices operate in coordination to simultaneously capture input signals across distinct sensing modalities during ongoing user interaction, and wherein the system operates as a continuous closed-loop architecture in which each executed adaptive action shapes subsequent input signals monitored across the two or more user devices, enabling ongoing adaptation of emotional state classifications and system responses without requiring explicit user input.

2-6. (canceled)
